(19) United States
(12) Patent Application Publication
(10) Pub. No.: US 2023/0181795 A1
(43) Pub. Date: Jun. 15, 2023

(54) METHOD OF MANUFACTURING A THREE-DIMENSIONAL MEDICAL DEVICE AND RESULTING MEDICAL DEVICE

(71) Applicant: PH TECH, LE TOURNE (FR)

(72) Inventors: Anthony PERES, DOMPIERRE SUR MER (FR); Guillaume HOFMANSKI, MIEUXCE (FR)

(21) Appl. No.: 17/924,755

(22) PCT Filed: May 12, 2021
(86) PCT No.: PCT/EP2021/062604

§ 371 Date: Nov. 11, 2022

(30) Foreign Application Priority Data
May 12, 2020 (FR) FR2004641

(51) Int. Cl. A61L 27/36; A61L 27/34

(52) U.S. Cl. CPC A61L 27/362; A61L 27/3691; A61L 27/3687; A61L 27/34; A61L 2430/04; A61L 2420/02

(57) ABSTRACT

The invention relates to a method for manufacturing a three-dimensional medical device from an acellular biological matrix characterized in that it comprises at least the following sequence of steps:

a/ wetting an acellular biological matrix with a liquid, and

b/ shaping the wet acellular biological matrix so that it takes on a three-dimensional shape.

The invention also relates to the obtained three-dimensional medical device and its use as an implant.

### TECHNICAL FIELD

The invention relates to a method for obtaining a three-dimensional medical device from an acellular biological matrix, as well as to the medical device obtained and its use in particular as an implant.

### PRIOR ART

Many surgical applications require soft tissue reinforcement and/or replacement of lost anatomical sections such as breast reconstructions.

These interventions require the addition of medical devices combining mechanical strength, flexibility, and biological compatibility.

To this end, biological matrices are used more and more for the manufacture of medical devices on account of their biological compatibility. Indeed, in breast reconstruction after a mastectomy or other cosmetic breast reconstruction surgery, many implants include an acellular dermal matrix that covers the implant/prosthesis and is connected to the pectoralis major muscle, thus avoiding prior stretching of the skin and muscles before implant placement. Furthermore, such a matrix helps to support the tissues and their regeneration.

However, for surgical indications requiring a particular three-dimensional shape, the use of an acellular biological matrix is only possible in the presence of a support allowing the biological matrix to be maintained in three dimensions. Moreover, current techniques are complex and require either perforating the acellular biological matrix to allow it to take on a three-dimensional shape, or cutting out elements of the matrix to assemble them by suturing, gluing, etc.

There is therefore a need for biological implants comprising an acellular biological matrix and that do not require the presence of a support to maintain the biological matrix in three dimensions, nor the use of techniques involving perforation or cutting, suturing, or gluing.

The object of the invention is to meet this need.

### SUMMARY OF THE INVENTION

To this end, the subject of the invention is a method for manufacturing a three-dimensional medical device from an acellular biological matrix, characterized in that it comprises at least the following sequence of steps:
a/ wetting an acellular biological matrix with a liquid, and
b/ shaping the wet acellular biological matrix so that it takes on a three-dimensional shape.

Advantageously, the wetting step makes the acellular biological matrix soft and malleable, allowing it to be shaped in three dimensions. Thus, the method makes it possible to obtain a three-dimensional medical device easily, quickly, and at reduced cost, which is usable as an implant without the need for a support for maintaining the three-dimensional biological matrix, nor for perforating the biological matrix.

A subject of the invention is therefore also a three-dimensional (non-planar) medical device comprising at least one three-dimensional acellular biological matrix, as well as its use as an implant.

Other features and advantages will emerge from the detailed description of the invention that follows.

### FIGURES

FIGS. 1a, 1b, and 1c are different views of an example of a hydrated acellular dermal matrix arranged on a form and held on said form by a disk or a counter-form, as described in Example 12.

### DETAILED DESCRIPTION OF THE INVENTION

### Definitions

Within the meaning of the invention, the term “acellular” biological matrix is intended to refer to a biological matrix in which the cellular elements have been eliminated through a decellularization process with the aim of destroying and/or removing the cells and their components from the extracellular matrix of the biological matrix while maintaining its structure and properties. In fact, in order for a biological matrix to be implanted in a recipient, it must be decellularized so as to decrease its immunogenicity.

Within the meaning of the invention, the term “allograft” is understood to refer to a biological matrix—a graft—originating from a donor belonging to the same biological species as the recipient.

Within the meaning of the invention, the term “shape” or “shaping” is understood to refer to obtaining a shape similar to that of another object considered as a model as a reference point.

Within the meaning of the invention, the term “implant” is understood to refer to a medical device used in surgery.

Within the meaning of the invention, the term “biological matrix” is understood to refer to a biomaterial derived from the human or animal species.

Within the meaning of the invention, the term “P4HB” is understood to refer to poly-4-hydroxybutyrate, a specific PHA. It is a homopolymer of a 4-hydroxybutyrate unit.

The term “peel test” is used here to refer to a test making it possible to determine the strength of adhesion between two materials. Each material is placed in pneumatic jaws at a given pressure and then separated at constant speed as specified in the examples.

Within the meaning of the invention, the term “PHA” is understood to refer to polyhydroxyalkanoates, which are biodegradable polyesters.

“Three-dimensional” is understood to refer to any object, in particular any medical device, in particular any implant, which is not flat.

Within the meaning of the invention, the term “solution” is understood to refer to a homogeneous mixture resulting from the dissolution of one or more solutes in a solvent.

Within the meaning of the invention, the term “suture retention force” or “suture retention strength” is understood to refer to a test for determining the force (N) required to pull a suture out of a specimen.

Within the meaning of the invention, the term “uniaxial tensile strength” is understood to refer to a test for determining the tension prior to the rupturing of the specimen being tested. The properties measured are ultimate tensile strength, breaking strength, and elongation at rupture.

Within the meaning of the invention, the term “viscosity” is understood to refer to a property of resistance to the flow of a fluid for non-turbulent flow.

Within the meaning of the invention, the term “xenograft” is understood to refer to a biological matrix—a graft—originating from a donor belonging to a biological species different from that of the recipient.

### Method for Manufacturing a Three-Dimensional Medical Device

According to a first aspect, the invention relates to a method for manufacturing a three-dimensional medical device from an acellular biological matrix.

Acellular biological matrices make up a large class of biomaterials extracted from grafts of various origins.

Preferably the biological matrix in the medical device according to the invention is of human or non-human animal origin (Allograft or Xenograft).

According to a particularly suitable embodiment, it is a biological matrix selected from among biological matrices of non-human animal origin selected from among biological matrices of porcine, bovine, equine, caprine, or fish origin, and mixtures thereof.

The biological matrix according to the invention may be selected from among all animal and/or human biological matrices, preferably from one of the following biological matrices: dermis, intestinal submucosa, aorta, bladder, amniotic membrane, peritoneum, pericardium, dura mater, tendons, cartilage, and mixtures thereof.

The biological matrix of the three-dimensional medical device according to the invention is acellular. There are many known methods for obtaining an acellular biological matrix. The methods used may be enzymatic and/or based on chemical solutions and/or on mechanical processes. The method used must be a method that makes it possible to obtain an acellular biological matrix that is capable of being used in surgery, particularly for the reconstruction of soft tissues.

The acellular biological matrix according to the invention is preferably an acellular biological matrix that has at least one of the following characteristics, and even more preferably all of them:
uniaxial tensile strength greater than or equal to 5 N/mm² for a 5 mm×50 mm specimen with a thickness taken into account in the calculation. Each end is secured in pneumatic grips lengthwise 1 cm from each edge. The edge spreading rate is 30 mm/min. Uniaxial tensile strength is found by dividing the maximum force (N)/(5 (mm)×thickness (mm)).
suture retention force greater than or equal to 5N for a 1 cm×1 cm sample using an appropriate suture (4-0 polypropylene-type) threaded 3 mm from the edge of the sample in its center, the opposite side being clamped in a pneumatic grip at approximately 5 mm. The two ends of the suture are clamped in the lower grip. The grip separation speed is 20 mm/min. The maximum force (N) is noted.

Preferably, the biological matrix used has the specifications defined in the applicable standards (“USP official monographs” latest version, ASTM) according to the type of biological matrix used.

The acellular biological matrix used for the implementation of the method according to the invention may be of various shapes. Preferably it:
is dry, preferably with a residual moisture content of between 10% and 18%, and/or
has a surface appearance that improves the adhesion of at least one polymer, in particular the adhesion of at least one polymer solution.

The acellular biological matrix used for the implementation of the method according to the invention may have different shapes (round, square, circular, irregular, etc.), be flat or in relief, have channels, have one of the surfaces textured, have regular or irregular thickness.

The method according to the invention for manufacturing a three-dimensional medical device from an acellular biological matrix comprises at least the following sequence of steps:
a/ wetting an acellular biological matrix with a liquid, preferably water, and
b/ shaping the wet acellular biological matrix so that it takes on a three-dimensional shape.

The purpose of step a/, which consists of wetting the acellular biological matrix, is to make said acellular biological matrix soft and malleable so as to allow its subsequent shaping.

Preferably, step a/ consisting of wetting the acellular biological matrix is performed by soaking the acellular biological matrix in a volume of water that allows for its immersion. Preferably the soaking is carried out for a period of 30 seconds to 20 minutes.

The water may be of different qualities (pyrogen-free, purified, etc.). According to a particularly suitable embodiment, step a/ is performed with pyrogen-free water.

Preferably, during step a/ of wetting, the acellular biological matrix becomes gray, i.e., glassy; more particularly it changes from white to gray (glassy).

After wetting, the acellular biological matrix may be shaped in three dimensions. Preferably step b/ of shaping is carried out by vacuum forming and/or wet molding and/or pressing.

In the case of shaping by vacuum forming, the matrix is placed on the desired shape or mold. The assembly is then placed in a system that allows the matrix to be held on the mold, forming it for the time required for the vacuum forming treatment. Said system may be composed of a food-grade vacuum bag of variable thickness (80 to 200 microns) and/or a solid or perforated counter-form or simply one with sections similar to the form (base) maintained on the matrix formed by any means during and after treatment. The assembly: “form +formed matrix” and holding system are then placed in a drying system (freeze-dryer, oven, etc.).

In a preferred embodiment the wetted biological matrix is placed on a perforated form (or in a mold). The assembly is then placed in a vacuum bag for treatment in a Henkelman-type vacuum machine. The vacuum is then produced over a cycle of 10 to 60 seconds between −1 bar and −2 bars, preferably −1 bar. The treatment time/vacuum rate combination may be assessed by the person skilled in the art depending on the desired result. The matrix is then shaped and maintained in this state by the vacuum bag.

In another embodiment, the biological matrices may be pressed in a hydraulic press over a period of 30 to 60 s, at between 6 bar and 200 bar, in particular between 50 and 200 bar, and at a temperature between 50° C. and 190° C., in particular between 50° C. and 100° C. Planar biological matrices (and consequently the planar medical devices according to the invention) are treated in a hydraulic press between two plates. Biological matrices (and consequently the medical devices according to the invention) in relief (biological implant-type treated by said method in the form of breast implants in anatomical or hemispherical shapes) may be pressed in molds having the desired imprints (of the type “molding machine for fabric cup”). In this specific case, the wet matrix is placed on a form then a counter-form is positioned above so that the matrix is “sandwiched” between the two forms. A temperature/pressure combination in the above quantities is applied for 30 to 60 s. The assembly is maintained after pressing to allow drying either by hot air oven or preferably by freeze-drying so as to obtain a desired relative humidity. The form and counter-form matrix assembly may also be freeze-dried before the application of the pressure/temperature combination so as to allow it to take shape. Said combination is then applied to mark the assembly and allow the 3D shape (in particular breast) to be maintained after hydration.

Preferably a drying method which does not denature the collagen by excessively high temperatures is preferred. The vacuum bag containing the form and the shaped matrix is then placed in a freeze-dryer for drying. Preferably, said bag may be placed in a freezer at −40° C. for freezing. After freezing, the formed matrix and the form are placed in a freeze-dryer for drying. The freeze-drying cycles of biological matrices are known to those skilled in the art. The matrix may also be taken out of the bag and placed with the form in a freeze-dryer for drying, the freeze-drying cycle then includes a freezing step.

In the case of pressing, the matrix is placed in a Short Nose Evolution-type press from Maugin. The machine is equipped beforehand with a form on the lower part and a counter-form on the piston. The operation then consists of pressing the biological matrix between the two elements for a defined time (between 30 s and 120 s) and at a chosen pressure (between 6 and 200 bars, in particular between 50 bars and 200 bars). The temperature may also be regulated (between 50° C. and 190° C., preferably between 50° C. and 150° C., in particular between 50° C. and 100° C.). The matrix thus formed is then dried on a suitable form.

In the case of wet casting, the die is placed on a form and then shaped on the form using a die setter. The piece is held on the form by mechanical fasteners (stapling, etc.).

According to a variant of the invention, the method for manufacturing a medical device according to the invention also comprises:
before or after step a/ or before or after step b/, a step for preparing the acellular biological matrix so as to allow adhesion to said acellular biological matrix of a solution comprising at least one polymer, and
before or after step a/ or before or after step b/, a step of partial or total coating of the acellular biological matrix with a solution comprising at least one polymer. The coating step only takes place after the step of preparing the acellular biological matrix so as to allow the adhesion on said acellular biological matrix of the solution comprising at least one polymer used for the coating.

According to a first embodiment of this variant, the method for manufacturing a medical device according to the invention comprises at least the following sequence of steps:
1/ preparing the acellular biological matrix so as to allow the adhesion on said acellular biological matrix of a solution comprising at least one polymer,
2/ coating the acellular biological matrix in whole or in part with a solution comprising at least one polymer,
3/ wetting the acellular biological matrix with a liquid,
4/ shaping the wetted and coated acellular biological matrix so that it takes on a three-dimensional shape.

According to a second embodiment of this variant, the method for manufacturing a medical device according to the invention comprises at least the following sequence of steps:
1/ preparing the acellular biological matrix so as to allow the adhesion to said acellular biological matrix of a solution comprising at least one polymer,
2/ wetting the acellular biological matrix with a liquid,
3/ coating the acellular biological matrix in whole or in part with a solution comprising at least one polymer,
4/ shaping the wetted and coated acellular biological matrix so that it takes on a three-dimensional shape.

According to a third embodiment of this variant, the method for manufacturing a medical device according to the invention comprises at least the following sequence of steps:
1/ preparing the acellular biological matrix so as to allow the adhesion to said acellular biological matrix of a solution comprising at least one polymer,
2/ wetting the acellular biological matrix with a liquid,
3/ shaping the wet acellular biological matrix so that it takes on a three-dimensional shape,
4/ coating the acellular biological matrix in whole or in part with a solution comprising at least one polymer.

According to a fourth embodiment of this variant, the method for manufacturing a medical device according to the invention comprises at least the following sequence of steps:
1/ wetting the acellular biological matrix with a liquid,
2/ preparing the acellular biological matrix so as to allow the adhesion to said acellular biological matrix of a solution comprising at least one polymer,
3/ coating the acellular biological matrix in whole or in part with a solution comprising at least one polymer.
4/ shaping the wetted and coated acellular biological matrix so that it takes on a three-dimensional shape.

According to a fifth embodiment of this variant, the method for manufacturing a medical device according to the invention comprises at least the following sequence of steps:
1/ wetting the acellular biological matrix with a liquid,
2/ preparing the acellular biological matrix so as to allow the adhesion to said acellular biological matrix of a solution comprising at least one polymer,
3/ shaping the wet acellular biological matrix so that it takes on a three-dimensional shape.
4/ coating the acellular biological matrix in whole or in part with a solution comprising at least one polymer.

According to a sixth embodiment of this variant, the method for manufacturing a three-dimensional medical device according to the invention comprises at least the following sequence of steps:
1/ wetting the acellular biological matrix with a liquid,
2/ shaping the wet acellular biological matrix so that it takes on a three-dimensional shape,
3/ preparing the acellular biological matrix so as to allow the adhesion to said acellular biological matrix of a solution comprising at least one polymer,
4/ coating the acellular biological matrix in whole or in part with a solution comprising at least one polymer.

Preferably, the step of preparing the cellular biological matrix consists of a chemical and/or mechanical and/or electrochemical and/or physical surface treatment. For example, it may be a treatment by abrasion and/or milling and/or microtexturing and/or laser and/or UV. The acellular biological matrix before coating must be prepared in such a way as to allow the adhesion of a polymer solution.

In a preferred embodiment, the acellular biological matrix prior to coating and/or after coating, should be dry or dried in such a way as to have a residual moisture content in the range of 10% to 18%. The residual moisture content is preferably measured using a Mettler Toledo brand halogen moisture analyzer-type desiccator.

One drying technique used is preferably that of Loss on Drying described in USP 41 (bovine dermis scaffold):
1 an empty aluminum dish is positioned in the device and taring is carried out,
2 the dish is filled with a sample of 1.0 g +/− 0.2 g cut into 4 mm² pieces,
3 the heating program at 130° C. is launched,
4 when the weight no longer varies over a given time, the result is displayed in %.

Another conventional oven-drying method may also be used. In this context, an aluminum dish previously weighed empty is filled with a sample of 5.0 g +/− 0.2 g cut into 4 mm² pieces. The entire thing is heated at 100° C. for 16 h. It is then weighed and the loss on drying is calculated:
dry matter %=[(weight of the dry extract+dish (g)−weight of dish (g))/g of sample]×100
moisture %=100−dry matter %.

The prepared biological matrix is then used for the application of a polymer solution coating.

The polymer or polymers used for coating the acellular biological matrix in the method according to the invention are preferably selected from among the following polymers: poly(glycolides), poly(lactide-co-glycolides); poly(lactic acid), poly(glycolic acid), poly(lactic acid-co-glycolic acids), polycaprolactones, poly(orthoesters), polyanhydrides, poly(phosphazenes), polyhydroxyalkanoates (including, in particular, P4HB and poly-3-hydroxybutyrate-co-3-hydroxy valerate (PHBV)), polyesters, poly(lactide-co-caprolactones), polycarbonates, tyrosine polycarbonates, polyamides, polyesteramides, poly(dioxanones), poly(alkylene alkylates), polyethers, polyvinyl pyrrolidones or PVP, polyurethanes, polyether esters, polyacetals, polycyanoacrylates, poly(oxyethylene)/poly(oxypropylene) copolymers, polyacetals, polyketals, polyphosphates, polyphosphoesters, polyalkylene oxalates, polyalkylene succinates, poly(maleic acids), chitin, chitosan, and mixtures thereof.

Preferably, the solution comprising at least one polymer was obtained previously through solubilization of the dry polymer(s) in at least one solvent, preferably at least one polar solvent.

In fact, the polymer(s) must be preferably converted into a solution that may enable coating using the appropriate solvent.

When the polymer is a PHA and, in particular, P4HB and/or one of its copolymers, the solvent is preferably selected from among the following polar solvents: dichloromethane, chloroform, tetrahydrofuran, dioxane, acetone, and mixtures thereof.

In a preferred embodiment, the P4HB is dissolved in an acetone solution, preferably in a ratio of from 5% to 25% (w/w). The respective amounts are combined, heated, and maintained at a temperature below the boiling point of acetone (approximately 56° C.) until the P4HB dissolves completely and the desired viscosity is obtained, at least 10%, more preferably 15%, and even more preferably 20% (w/w).

Preferably, after dissolving the polymer(s) in a solvent, the polymer solution is degassed and/or debubbled in order to purge the mixture of air bubbles. Preferably, the solution is placed under vacuum (minimum −1 bar) for the time required for complete degassing/boiling.

The step of coating the acellular biological matrix with a polymer solution is preferably carried out at a temperature less than or equal to the denaturation temperature of the collagen. In a particularly suitable manner, the coating is carried out at a temperature between 10 and 60° C., preferably between 10 and 50° C., even more preferably between 20 and 50° C.

The coating may be performed by any suitable means, preferably by solvent casting, spray coating, or dip coating.

Advantageously, the method according to the invention allows for the direct application of a polymer solution at the desired concentration to a previously prepared acellular biological matrix, and it is not necessary to make a polymer film or sheet beforehand and then place it on the support in order to allow adhesion of the elements to each other by heating.

When it is carried out by “solvent casting,” the coating may be carried out using different technologies: knife, double side, commabar, case knife, engraved roller, 2 rollers, 3 roller combi, microroller, 5 rollers, reverse roller, rotary screen, dipping, slot die, curtain coating, or hot-melt slot die. The simplest method is based on the use of an Elcometer-type casting knife. The biological matrix is placed on a table. The quantity of polymer solution required as a function of the desired thickness and the surface to be treated is placed on the acellular biological matrix. The gardener knife is then moved over the acellular biological matrix in order to make the coating on the implant uniform. The gardener knife has been adjusted beforehand to a certain height.

In order to automate the operation, a coating machine may be used. The polymer solution is pumped through a slot die in order to be applied to the moving biological implant. In a preferred embodiment, the width of the slot die is 600 mm, and the conveyance speed of the implant is from 1-10 m/min. The pumping speed, the conveyance speed, the width of the slot die, and the concentration of the solution may be adjusted in order to obtain an implant with a coating of the desired thickness and width.

When the coating is carried out by spray coating or atomization, the polymer solution is pumped up to a nozzle that projects droplets onto the surface to be treated. This technique is particularly advantageous for the treatment of biological matrices having 3D shapes (example: biological implant having a hemispherical, ovoid, tubular shape, or in the form of anatomical breast implants, etc.).

When the coating is performed by means of dip coating, or immersion, the piece to be treated is dipped in a dissolved, melted, softened, or fluidized powder material in order to cover it with a layer of that material. This technique is particularly advantageous for the treatment of raised and planar biological implants.

After coating, the medical device obtained is composed of a layer of biological matrix that is coated in whole or in part with at least one polymer in solution, and the entire thing is then dried and then preferably pressed in order to obtain a uniform thickness.

In a preferred embodiment, the acellular biological matrices that are coated with the polymer solution are placed in a furnace or oven or heating chamber in order to allow complete evaporation of the solvent. The temperature is set between 0° C. and 50° C., between 15 and 40° C., preferably 30° C. plus or minus 5°, so as to prevent excessively rapid evaporation and deformation of the biological matrix.

According to a particular variant of the invention, the acellular biological matrices that have been coated or not with the polymer solution, optionally before or after drying, are pressed in a hydraulic press for a duration of 30 to 60 s, at between 6 bar and 200 bar, in particular at between 50 and 200 bar and at a temperature between 50° C. and 190° C., in particular between 50° C. and 100° C. Planar biological matrices (and consequently the planar medical devices according to the invention) are treated in a hydraulic press between two plates. Biological matrices (and consequently the medical devices according to the invention) in relief (biological implant-type treated by said method in the form of breast implants in anatomical or hemispherical shapes) may be pressed in molds having the desired imprints (of the type “molding machine for fabric cup”). In this specific case, the wet matrix is placed on a form then a counter-form is positioned above so that the matrix is “sandwiched” between the two forms. A temperature/pressure combination in the above quantities is applied for 30 to 60 s. The assembly is maintained after pressing to allow drying either by hot air oven or preferably by freeze-drying so as to obtain a relative humidity as defined above. The form and counter-form matrix assembly may also be freeze-dried before the application of the pressure/temperature combination so as to allow it to take shape. Said combination is then applied to mark the assembly and allow the 3D shape (in particular breast) to be maintained after hydration. The shaping described above may be carried out on a coated or uncoated implant such that the matrix formed alone may be coated after taking shape according to the methods described in the above sections.

### Three-Dimensional Medical Device and Uses

According to a second aspect, the invention also specifically refers to a three-dimensional medical device, capable of being obtained by implementing the method according to the invention, comprising at least one three-dimensionally shaped acellular biological matrix.

Advantageously, the three-dimensional medical device according to the invention does not comprise a support for maintaining the acellular biological matrix in three dimensions, said biological matrix having been shaped in three dimensions by shaping processes carried out on the previously wet (preferably immersed in water) acellular biological matrix.

Preferably, the three-dimensional medical device according to the invention comprises at least one acellular biological matrix that is covered in whole or in part by at least one layer comprising at least one polymer.

The three-dimensional medical device according to the invention may comprise one or more acellular biological matrices, one or more layers of polymer(s), and optionally other constituents. For example, the three-dimensional medical device according to the invention may comprise at least one acellular biological matrix that is covered in whole or in part by at least two layers comprising at least one polymer.

According to a particular embodiment, the three-dimensional medical device consists exclusively of an acellular biological matrix that is covered in whole or in part by a layer comprising at least one polymer.

According to another particular embodiment, the three-dimensional medical device consists exclusively of an acellular biological matrix that is covered in whole or in part by two layers comprising at least one polymer.

The presence of the polymer(s) in the three-dimensional medical device according to the invention enables optimal reinforcement to be achieved during tissue reconstruction supported by the biological matrix, even in the event of infection. In fact, the presence of at least one polymer with the acellular biological matrix makes it possible to render the biological matrix resistant to infections while retaining the qualities of the biological matrix in terms of mechanical resistance, flexibility, and biological compatibility. It may thus be used for medical applications, particularly in surgery.

The polymer present in the three-dimensional medical device according to the invention may be any type of polymer suitable for use as a medical device and in particular as an implant, especially as a biological implant, in particular as a surgical implant or surgical biological implant.

The polymer or polymers used that are present in the three-dimensional medical device according to the invention are preferably selected from among the following polymers: poly(glycolides), poly(lactide-co-glycolides); poly(lactic acid), poly(glycolic acid), poly(lactic acid-co-glycolic acids), polycaprolactones, poly(orthoesters), polyanhydrides, poly(phosphazenes), polyhydroxyalkanoates (including, in particular, P4HB and poly-3-hydroxybutyrate-co-3-hydroxy valerate (PHBV)), polyesters, poly(lactide-co-caprolactones), polycarbonates, tyrosine polycarbonates, polyamides, polyesteramides, poly(dioxanones), poly(alkylene alkylates), polyethers, polyvinyl pyrrolidones or PVP, polyurethanes, polyether esters, polyacetals, polycyanoacrylates, poly(oxyethylene)/poly(oxypropylene) copolymers, polyacetals, polyketals, polyphosphates, polyphosphoesters, polyalkylene oxalates, polyalkylene succinates, poly(maleic acids), chitin, chitosan, and mixtures thereof.

According to a particularly suitable embodiment, the three-dimensional medical device according to the invention comprises at least one PHA, and even more preferably at least one PHA that is selected from among P4HB, the copolymers of P4HB, and mixtures thereof. PHAs are a family of materials produced by many microorganisms. One noteworthy example is U.S. Pat. No. 6,316,262 of Metabolix, Inc. from Cambridge, Mass., USA, which describes a method for obtaining a biological system that enables the production of polyhydroxyalkanoate polymers containing 4-hydroxy acids. U.S. Pat. Nos. 6,245,537, 6,623,748, 7,244,442, and 8,231,889 also describe methods for preparing PHAs that are suitable for medical use and for medical devices according to the invention.

Preferably, PHA and, in particular, P4HB and/or copolymers thereof have a low level of endotoxins; in particular, they make it possible for levels of below 20 EU per medical device to be achieved.

The polymer layer(s) may comprise one or more channels that make it possible, during use in surgery, to incorporate elements promoting optimal construction of the tissues in which the device is used as an implant (PRP, stem cells, antibiotics, etc.). These channels may be circular or non-circular. They preferably have an internal surface of between 0.007 mm² and 0.8 mm². They may be obtained by imprinting a form in the biological matrix through pressing before coating of the biological matrix. The form is then removed after coating and drying.

The medical device according to the invention is preferably used as a biological implant, in particular as a surgical implant or a surgical biological implant. According to one variant, it is an implant for breast reconstruction.

In particular and without limitation, the three-dimensional medical devices according to the invention may be used for the following applications: repair, regeneration, and replacement of soft and hard tissues, healing device, bandage, patch, dressing, dressing for burns, dressing for ulcers, skin substitute, hemostatic, tracheal reconstruction device, organ rescue device, dural substitute, dural patch, nerve guide, nerve regeneration or repair device, hernia repair device, hernia mesh, hernia plug, temporary wound or tissue support device, tissue engineering scaffolding, guided tissue repair/regeneration device, mesh fixation devices, non-stick membrane, adhesion barrier, tissue separation membrane, retention membrane, sling, pelvic floor reconstruction device, urethral suspension device, urinary incontinence treatment device, bladder repair device, bulking or filling device, rotator cuff repair device, meniscus repair device, meniscus regeneration device, guided tissue regeneration membrane for periodontal tissue, anastomosis device, cell-seeded device, cell encapsulation device, controlled release device, drug delivery device, plastic surgery device, breast lift device, mastopexy device, breast reconstruction device, breast augmentation device, breast reduction device, breast reconstruction devices after mastectomy with or without breast implants, rhinoplasty device.

The invention is now illustrated by examples and test results.

### EXAMPLES AND TEST RESULTS

### Example 1

### Wetting and Shaping

An acellular dermal matrix is wetted as follows: it is immersed in a volume of type 2 purified water for 15 min.

It is then placed on an anatomical breast form, said form being screwed onto a perforated plate. The dermal matrix is attached to the edges of the plate by mechanical fasteners (staples, peripheral frame, etc.). The assembly is placed in an 80 micron vacuum bag. The treatment is carried out on a Henkelman Marlin-type machine. The vacuum treatment is carried out at −1 bar. After treatment, the assembly is placed in a freezer at −40° C. Once frozen, the contents are taken out of the bag and then placed in a freeze-dryer for drying.

Preferably the matrix is sterilized.

The acellular biological matrix thus shaped in three dimensions may be used as a medical device, in particular as an implant, notably in surgery.

### Example 2

### Wetting and Shaping

A dermal matrix is wetted as follows: it is immersed in a volume of pyrogen-free water for 3 min.

It is then placed in a Short Nose Evolution-type press from Maugin. A pressure is exerted at 100 bars for 30 s and at a temperature of 100° C. The matrix thus treated is placed in an oven for drying at 40° C. overnight.

After drying, the implants thus formed may be pressed in order to standardize the thickness in a hydraulic press equipped with the appropriate form and counter-form. The space between the two is adjusted so as to control the desired thickness. The pressing time varies between 30 s and 60 s and the pressure between 50 bars and 200 bars.

The acellular biological matrix thus shaped in three dimensions may be used as a medical device, in particular as an implant, notably in surgery.

### Example 3

### Treatment of the Acellular Biological Matrix

A dry, planar, acellular dermal matrix (residual moisture content between 10% and 18%) is placed on a numerically controlled machine tool. A carbide cutter measuring 5 mm in diameter is mounted on the machine tool. A rotation speed of from 20,000 to 40,000 rpm is used with an advancement of 2 m/min. The depth is variable depending on the desired final thickness. The surfacing may be total or concern only a portion of the implant so as to define forms.

The matrix is then coated with a solution of polymer(s), preferably according to Example 6.

The matrix is then wetted and shaped according to one of examples 1 or 2.

The matrix is then preferably dried so as to have a residual moisture content of between 10% and 18%.

### Example 4

### Treatment of the Acellular Biological Matrix

A dry, planar, acellular dermal matrix (residual moisture content between 10% and 18%) is placed on a brushing/carding machine. The surface of the implant is thus treated with cards of desired diameter (105 mm, for example) composed of metallic wires (having a diameter of 0.2 mm, for example).

The matrix is then coated with a polymer solution.

The matrix is then wetted and shaped according to one of examples 1 or 2.

The matrix is then preferably dried so as to have a residual moisture content of between 10% and 18%.

### Example 5

### Treatment of the Acellular Biological Matrix

A dry, acellular dermal matrix (residual moisture content between 10% and 18%) is treated with a Telea Biotech-type high-frequency electrical field (4-64 MHz) so as to create cavities and/or perforations 0.6 mm in diameter.

The matrix is then coated with a polymer solution.

The matrix is then wetted and shaped according to one of examples 1 or 2.

The matrix is then preferably dried so as to have a residual moisture content of between 10% and 18%.

### Example 6

### Treatment of the Acellular Biological Matrix

An acellular dermal matrix is treated as specified in example 3. A 2 mm carbide cutter is mounted on the machine tool in order to draw in the matrix channels having a sinusoidal shape along the length of the matrix. Speed and advancement are similar to example 1. The channels are repeated at regular intervals across the width in order to cover the implant all or in part. The depth of the channels depends on the desired final diameter. In another embodiment, an impression may be made so as to press the channels into the matrix.

The matrix is then coated with a polymer solution.

The matrix is then wetted and shaped according to one of examples 1 or 2.

The matrix is then preferably dried so as to have a residual moisture content of between 10% and 18%.

### Example 7

### Example of a Solvent Casting Coating Method

An acellular dermal matrix treated according to example 3 having the dimensions 2 cm×6 cm, or 12 cm² is placed on a table. Since the desired amount of P4HB is 0.0164 g/cm², an 18% P4HB/acetone solution (w/w) is prepared. The required amount is removed and deposited on the implant. The casting knife is then moved translationally on the implant in order to make the thickness of the coating uniform. A coating machine may be used to automate the operation. An acetone/P4HB solution is prepared in order to supply the machine. The solution is pumped through a slot die in order to be applied to the moving planar biological implant. In a preferred embodiment, the width of the slot die is 600 mm, the conveyance speed of the implant is from 1-10 m/min.

The matrix is then wetted and shaped according to one of examples 1 or 2.

The matrix is then preferably dried so as to have a residual moisture content of between 10% and 18%.

### Example 8

### Example of the Dip Coating Process

An acellular biological matrix treated according to example 4 measuring 4 cm×6 cm is immersed by a machine (the implant is placed on a vertical crosspiece) into a tank containing an acetone/P4HB solution at the desired concentration. The biological matrix is then extracted from the tank by an upward vertical movement at a speed of 25 mm/min. The density of P4HB obtained by coating is then from 0.02 to 0.04 g/cm². The parameters (size of the part to be treated, speed of the crosspiece, concentration of the solution) that are required to obtain the desired coating are ultimately conceivable by those skilled in the art.

The matrix is then wetted and shaped according to one of examples 1 or 2.

The matrix is then preferably dried so as to have a residual moisture content of between 10% and 18%.

### Example 9

### Example of Drying and Pressing and Characteristics of the Medical Devices Obtained

A dermal matrix treated according to example 3 is coated with approximately 0.03 g/cm² of P4HB and is pressed for 30 s at 100 bar of pressure and at 50° C. (previously heated press plates). On three test pieces tested, with an average thickness of 1.35 mm, the maximum uniaxial tensile strength (UTS) is 26.50 MPa.

The test is carried out on an Instron measurement bench model 3342/L2345. The specimen is cut with a bone-shaped type V cutter as described in standard ASTM D-638-5. The piece cut in this manner is inserted by each end into the pneumatic grips of the bench (60 psi), leaving a central portion of 2.5 cm. A speed of 25 mm/min is applied until the piece breaks. The uniaxial tensile strength is noted (max. force/sectional area).

A T-peel test is carried out. Specimens of 2 cm×6 cm are cut out. The P4HB coating is separated from the biological implant on a section measuring 1.5 cm×2 cm. The two pieces separated in this manner are placed in the pneumatic grips (45 psi). The section of the coated biological implant is separated at a speed of 25 mm/min. The T-peel force is measured over a standard width of 20 mm and on an average of 5 peaks (loads). The adhesion was stronger over the three test pieces that were tested, the coated layer did not manage to be peeled in the test.

### Example 10

### Example of Drying and Pressing and Characteristics of the Medical Devices Obtained

Employing the same conditions as in the preceding tests, a dermal matrix that has been treated according to example 4 is coated with approximately 0.03 g/cm² of P4HB and then pressed for 60 s at 200 bar and 100° C. An average UTS max value is obtained over 3 test specimens of 27.65 MPa.

### Example 11

Employing the same conditions as in the preceding tests, a dermal matrix that has been treated according to example 5 is coated with approximately 0.03 g/cm² of P4HB and then pressed for 30 s at 100 bar and 100° C. An average UTS max value is obtained over 3 test specimens of 33.07 MPa.

### Example 12

### Example of Shaping the Matrix Before Coating

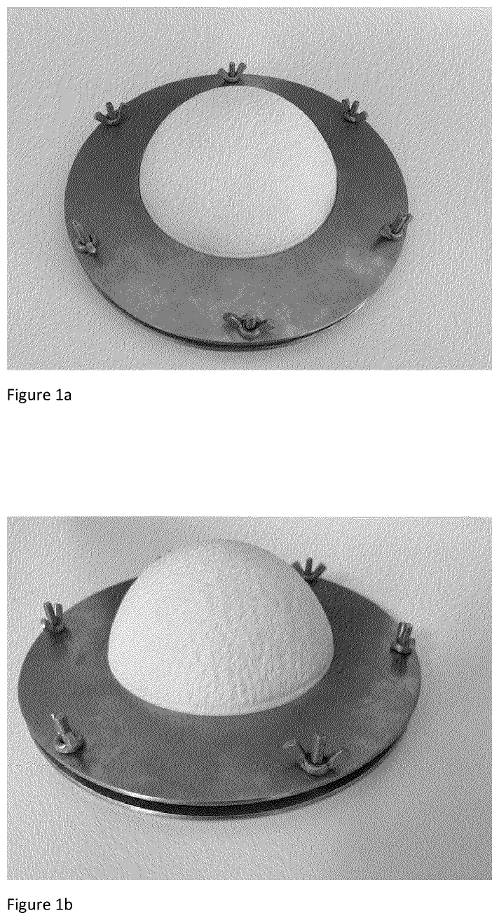

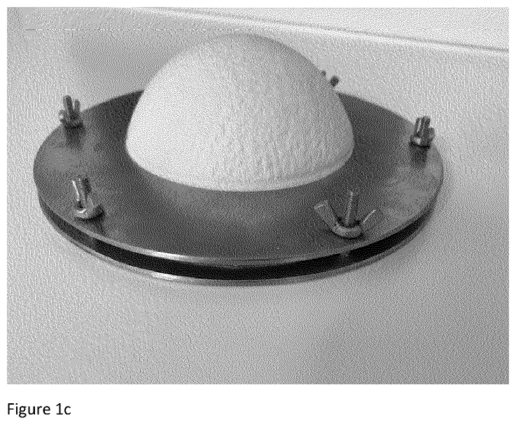

A dermal matrix is hydrated for 15 min in water. The matrix is then positioned on a form and held on said form by a disc or a counter-form (FIGS. 1a, 1b and 1c). The entire thing is freeze-dried to obtain a device with a residual moisture content of less than 18%. The matrix is then positioned on a press for pressing on a form at 6 bars at a temperature of 190° C. for 60 s. Over three test pieces tested, with an average thickness of 1.35 mm, the maximum average uniaxial tensile strength (UTS) is 26.50 MPa. The test is carried out on an Instron measurement bench model 3342/L2345. The specimen is cut with a bone-shaped type V cutter as described in standard ASTM D-638-5. The piece cut in this manner is inserted by each end into the pneumatic grips of the bench (60 psi), leaving a central portion of 2.5 cm. A speed of 25 mm/min is applied until the piece breaks. The uniaxial tensile strength is noted (max. force/sectional area).

According to a method of preparation described above in the preceding examples, the dermal matrix may then be treated so as to allow the adhesion of the P4HB to the shape thus created by the method also described in the preceding examples.

1. A method for manufacturing a three-dimensional medical device from an acellular biological matrix characterized in that it comprises at least the following sequence of steps:
a/ wetting an acellular biological matrix with a liquid, and
b/ shaping the wet acellular biological matrix so that it takes on a three-dimensional shape.

2. The method for manufacturing a medical device as set forth in claim 1, characterized in that step a/ consisting in wetting the acellular biological matrix is carried out by soaking the acellular biological matrix in a volume of water allowing it to be immersed.

3. The method for manufacturing a medical device as set forth in claim 2, characterized in that the soaking is carried out for a period of 30 seconds to 20 minutes.

4. The method for manufacturing a medical device as set forth in claim 1, characterized in that step a/ is carried out with pyrogen-free water.

5. The method for manufacturing a medical device as set forth in claim 1, characterized in that step a/ is carried out so as to obtain an acellular biological matrix gray in color.

6. The method for manufacturing a medical device as set forth in claim 1, characterized in that step b/ of shaping is carried out by vacuum forming and/or wet molding and/or pressing.

7. The method for manufacturing a medical device as set forth in claim 1, characterized in that the acellular biological matrix is of human and/or animal origin.

8. The method for manufacturing a medical device as set forth in claim 7, characterized in that the acellular biological matrix of animal origin is selected from among the acellular biological matrices of porcine, bovine, equine, caprine, or fish origin, and mixtures thereof.

9. The method for manufacturing a medical device as set forth in claim 1, characterized in that the acellular biological matrix is selected from one of the following biological matrices: dermis, intestinal submucosa, aorta, bladder, amniotic membrane, peritoneum, pericardium, dura mater, tendons, cartilage, and mixtures thereof.

10. The method for manufacturing a medical device as set forth in claim 1, characterized in that it comprises:
before or after step a/ or before or after step b/, a step of preparing the acellular biological matrix so as to allow the adhesion to said acellular biological matrix of a solution comprising at least one polymer, and
before or after step a/ or before or after step b/, and after the step of preparing the acellular biological matrix, a step of coating the acellular biological matrix in full or in part with a solution comprising at least one polymer.

11. The method for manufacturing a medical device as set forth in claim 10, characterized in that the coating is performed at a temperature lower than or equal to the denaturation temperature of the collagen.

12. The method for manufacturing a medical device as set forth in claim 11, characterized in that the coating is performed at a temperature between 10 and 60° C.

13. The method for manufacturing a medical device as set forth in claim 10, characterized in that before coating and/or after coating, the acellular biological matrix is dried so as to have a residual moisture content of between 10% and 18%.

14. The method for manufacturing a medical device as set forth in claim 10, characterized in that the step of preparing the wet cellular biological matrix so as to allow adhesion to said acellular biological matrix of a solution comprising at least one polymer, consists of a chemical and/or mechanical and/or electrochemical and/or physical surface treatment.

15. The method for manufacturing a medical device as set forth in claim 10, characterized in that the coating is carried out by solvent casting, spray coating, or dip coating.

16. The method for manufacturing a medical device as set forth in claim 10, characterized in that the solution comprising at least one polymer was obtained beforehand through solubilization of the polymer(s) in at least one polar solvent.

17. The method for manufacturing a medical device as set forth in claim 16, characterized in that, after solubilization of the polymer(s), the solution is degassed and/or debubbled under vacuum in order to purge the mixture of air bubbles.

18. The method for manufacturing a medical device as set forth in claim 10, characterized in that the solution of polymer(s) comprise one or more polymers selected from among the following polymers: polyhydroxyalkanoates, poly(glycolides), poly(lactide-co-glycolides), poly(lactic acid), poly(glycolic acid), poly(lactic acid-co-glycolic acids), polycaprolactones, poly(orthoesters), polyanhydrides, poly(pho sphazenes), polyhydroxyalkanoates, polyesters, poly(lactide-co-caprolactones), polycarbonates; tyrosine polycarbonates, polyamides, polyesteramides, poly(dioxanones), poly(alkylene alkylates), polyethers, polyvinyl pyrrolidones, polyurethanes, polyether esters, polyacetals, polycyanoacrylates, poly(oxyethylene), copolymers of poly(oxypropylene), polyacetals, polyketals, polyphosphates, polyalkylene oxalates, polyalkylene succinates, poly(maleic acids), chitin, chitosan, and mixtures thereof.

19. The method for manufacturing a medical device as set forth in claim 10, characterized in that the polymer solution comprises at least P4HB dissolved in an acetone solution.

20. A three-dimensional medical device, obtainable by implementing the method as set forth in claim 1, characterized in that it comprises at least one three-dimensionally shaped acellular biological matrix.

21. The three-dimensional medical device as set forth in claim 20, characterized in that it does not comprise a support for maintaining the biological matrix in three dimensions.

22. The three-dimensional medical device as set forth in claim 20, characterized in that the three-dimensionally shaped acellular biological matrix is covered in full or in part by at least one layer comprising at least one polymer.

23. The three-dimensional medical device as set forth in claim 20, characterized in that it is an implant for breast reconstruction.
